(19) United States
(12) Patent Application Publication
(10) Pub. No.: US 2008/0064598 A1
(43) Pub. Date: Mar. 13, 2008

(54) Mycorrhizal Fungi Preparation, their Production and their Use in the Inoculation of Plants

(75) Inventors: Michel De Rougemont, Kaiseraugst (CH); Meinolf Stuetzer, Jessnitz (DE); Angelika Spindler, Dessau (DE)

Correspondence Address: OBLON, SPIVAK, MCCLELLAND MAIER & NEUSTADT, P.C., 1940 DUKE STREET, ALEXANDRIA, VA, 22314, US

(73) Assignee: Mycosym International AG, Basel (CH)

(21) Appl. No.: 11/718,759

(22) PCT Filed: Nov. 4, 2005
(86) PCT No.: PCT/CH05/00648

§ 371 Date: Oct. 1, 2007

(30) Foreign Application Priority Data
Nov. 4, 2004 (CH) 1828/04

(51) Int. Cl. A01N 63/04; A01N 59/00; C12N 1/14

(52) U.S. Cl. 504/101; 424/93.5; 435/254.1; 504/117

(57) ABSTRACT

The invention relates to mycorrhizal fungi preparations that form arbuscles. To obtain said preparations, a raw inoculum is cultivated on a porous granulate and the raw inoculum that is produced is milled in such a way that the active spores are not destroyed. Said preparations are suitable for fertilisation and crop protection in agriculture and forestry. Glomus sp., and in particular Glomus intraradices are mycorrhizal fungi that are suitable for this application. A hammer/impact mill is used to mill the raw inoculum.

The present invention relates to arbuscular mycorrhizal fungal preparations which can be used for improving the inoculation of plants. The inoculation of plants with mycorrhizal fungi is a useful complementation of fertilization and crop protection in agriculture, in particular in the cultivation of vegetables fruits and flowers, and in silviculture.

Arbuscular mycorrhizal fungi (AM fungi) enter an advantageous symbiosis with a multiplicity of plants. As the result of the colonization of the root cells of the plant with such fungi, a more extensive root system with better reticulate fungal penetration develops. Thus, fungal hyphae continue growing outside the normal root ball into a zone in which nutrients such as in particular phosphorus are normally not accessible to the plant. It has been shown that AM fungi bring about a significant improvement of plant growth (cf., for example, S. Gianinazzi, H. Schüepp, J. M. Barea, K. Haselwandter “Mycorrhizal technology in Agriculture”, Birkhäuser 2002; E. Sieverding “Vesicular-Arbuscular Mycorrhiza Management in Tropical Agrosystems, GTZ 1991). Despite this economically useful property AM fungal preparations have not gained major importance in agriculture, in particular the cultivation of vegetables and fruits or in silviculture. One reason can partly be attributed to the lacking availability of substantial amounts of preparations which can be applied without problems AM fungi are obligatory phytotrophs which are not capable of multiplication without host plant. Their production therefore requires a combined system. Currently AM fungal inocula, in particular Glomus sp., are prepared by culturing host plants in pots or beds on inert substrates (cf., for example H. Baltruschat et al., EP 0 183 040, 1985). Alternative production methods under meristematic in-vitro conditions (J. A. Fortin et al., U.S. Pat. No. 5,554,530, 1996) or in what are known as aeroponic systems (D. M. Sylvia et al., U.S. Pat. No. 5,096,481) are also used.

Such cultivation methods have the advantage of being simple and make possible the production of a large amount of AM fungal spores. All porous and biologically inert materials are suitable as substrates for the cultivation. A relatively coarse particle size of approximately 1 to 8 mm and pore diameters in the same order of magnitude as the spore dimensions have proven to be particularly suitable. Expanded clay, expanded slate, volcanic rocks or perlite have these properties. The use of smaller particle sizes or of less porous materials results in lower process yields.

Mycorrhizing spores are developed within the free channels of each substrate grain. Typically, AM fungal preparations are produced with approximately 100-200 infectious units per milliliter (cf. Gianinazzi, above). Depending on the particle sizes of the substrate, these units are divided to provide only 5 to 20 granules per milliliter. Despite a high number of active spores and hyphae, it thus follows that the spatial distribution around the plant roots is unsatisfactory. The efficacious and economically justifiable application of AM fungal preparations thus remains limited.

The invention aims at solving the problem of the spatially limited application of AM fungal preparations.

In accordance with the invention, a crude inoculum is first prepared by culturing AM fungi, such as Glomus sp., in particular Glomus intraradices, on porous granules as substrate, as described by Baltruschat (see above). The porous granules used for this purpose are expediently expanded clay or attapulgite or expanded slate or volcanic rock or perlite or vermiculite or a mixture of two or more of these materials. The particle size of these porous granules is expediently approximately 1 and 6 mm. Expanded clay with a particle size of 1 to 4 mm and a bulk density of approximately 250 to 300 kg/m³, in particular 270 kg/m³, has proved to be particularly expedient.

The crude inoculum contains spores, hyphae and fine root residues of the host plants. An analytical biological test as described by Sieverding (see above) reveals a probable number of infectious units of typically 300 to 600 per gram. In highly productive units, this number may even be higher than 2000. Since these units are hidden in the pores of the substrate, no isolated spores of the AM fungi, such as Glomus sp., in particular Glomus intraradices, can be observed under the microscope.

In accordance with the invention, the crude inoculum is then comminuted carefully so that the active spores are not destroyed. This can be accomplished by using a grinding process which is based on impact and not on friction. Suitable types of mills which operate by impact are, in particular, hammer mills, bladed-disk mills or jet mills. The operative parameters of the mill must be adjusted in such a way that the particle size of the ground product is in the range of approximately 100-500 μm, preferably approximately 100-250 μm. This means that, in particular, the rotational speed must be adjusted accordingly and the discharge screen must be selected accordingly. In the ground product, up to 1000 free spores or spore agglomerations per gram can be counted under the microscope.

The comminuted product which is obtained in accordance with the invention can be used directly as solid for inoculating plants with AM fungi, or else it can be standardized to a nominal activity by dilution with a customary inert material. It can also be formulated together with customary formulation auxiliaries to give a water-suspendable preparation.

The invention and its advantages are illustrated in greater detail in the examples which follow.

### EXAMPLE 1

Comparison of Different Types of Mills, and Spore Counting

An AM fungal inoculum was prepared in expanded-clay granules with a particle size of 2-4 mm, proceeding as described by Baltruschat (see above).

The resulting mycorrhizal-containing product was ground in different mills under different conditions. The number of free spores or spore agglomerations in the mill base was determined under the microscope.

| | | | | | | Number of |
|---|---|---|---|---|---|---|
| | | | | | | free spores |
| | | | Screen | | | and spore |
| | | | fractions obtained | | | agglomera- |
| | Mill | | Particle | | Weight | tions |
| Sample | setting | | size | | fraction | per gram |
| Crude | | | <2 | mm | 3% | 0 |
| product | | | 2-4 | mm | 95% | |
| (granules) | | | >4 | mm | 2% | |
| Laboratory | Gap 1 | mm | 0-1 | mm | 100% | 0 |
| roll | | | | | | |
| crusher | | | | | | |
| Laboratory | | | 0-0.125 | mm | 46.6% | 85 |
| jaw crusher | | | 0.125-0.5 | mm | 23.0% | 84 |
| | | | >0.5 | | 30.4% | 12 |
| Roll | | | 0-0.125 | mm | 24.5% | 75 |
| crusher | | | 0.125-0.5 | mm | 22.0% | 129 |
| type 1 | | | >0.5 | | 53.5% | 14 |
| Roll | | | 0-0.125 | mm | 29.9% | 45 |
| crusher | | | 0.125-0.5 | mm | 30.7% | 186 |
| type 2 | | | >0.5 | mm | 39.3% | 48 |
| Pilot | Ø220 | mm | 0-0.125 | | 46.7% | 250 |
| hammer | 1400 | rpm | 0.125-0.5 | mm | 17.1% | 568 |
| mill No. 1 | screen: | | >0.5 | | 36.3% | 0 |
| | 2 | mm | | | | |
| Pilot | Ø200 | mm | Average | | 100% | 9 |
| hammer | 1500 | rpm | 36 μm | | | |
| mill No. 2 | screen: | | | | | |
| | 0.7 | mm | | | | |
| Pilot | Ø240 | mm | 0-0.125 | mm | 60.3% | 70 |
| bladed-disk | 4750 | rpm | 0.125-0.5 | mm | 36.1% | 120 |
| mill | screen: | | >0.5 | | 3.6% | 0 |
| | 2 | mm | | | | |

### EXAMPLE 2

Degree of Mycorrhization of the Roots of Test Plants (Tagetes “Orangenflamme”)

Plastic plant pots (round, height 6.5 cm, diameter: 9 cm at the top, 6 cm at the bottom) were filled with a mixture of, firstly, 500 mg or 1000 mg of the product ground in accordance with Example 1 with the Pilot hammer mill No. 1, or of the crude product of Example 1, and, secondly, a substrate consisting of potting compost (Floradur-Fein) and sterilized sand in the ratio 1:1. Pots filled only with substrate (potting compost/sand) acted as the control.

Two seedling plants were pricked out into each pot. The pots were watered normally with tap water and fertilized once per week with a solution of Wuxal top N (concentration 0.05% during the first 3 weeks thereafter 0.1%).

After 10 weeks, the entire root ball was stained and evaluated under the microscope for the presence of mycorrhiza-developing hyphas, arbuscules, vesicles and spores, using a scoring key (cf. MeGonigle, T. P., Miller, M. H., Evans, D. C., Fairchild, G. L. and Swan J. A., 1990. A new method which gives an objective measure of colonization of roots by vesicular-arbuscular mycorrhizal fungi. New Phytol. 115: 495 501).

With 5 replications, the following degrees of mycorrhization resulted:

Weighed-in quantity

| | 500 mg | 1000 mg |
|---|---|---|
| Control (substrate only) | 1.3% | 1.3% |
| Crude product (granules) | 17.5% | 11.3% |
| Pilot hammer mill No. 1 | 23.8% | 27.5% |

The above data show that the use of the ground product increased the degree of mycorrhization by 36% and almost 144% in comparison with the crude granules.

1. A process for the preparation of arbuscular mycorrhizal fungal preparations characterized in that a crude inoculum is cultured on porous granules and the resulting crude inoculum is comminuted carefully so that the active spores are not destroyed.

2. The process as claimed in claim 1, wherein the porous granules are selected from the group consisting of expanded clay, attapulgite, expanded slate, volcanic rock, perlite, vermiculite, and a mixture of two or more of these materials.

3. The process as claimed in claim 1, wherein the particle size of the porous granule substrate is between 1 and 6 mm.

4. The process as claimed in claim 1, wherein the porous granules are expanded clay having a particle size of from 1 to 4 mm and a bulk density of approximately 250 to 300 kg/m³.

5. The process as claimed in claim 4, wherein the expanded clay has a bulk density of 270 kg/³.

6. The process as claimed in claim 1, characterized in that the arbuscular mycorrhizal fungus is Glomus sp.

7. The process as claimed in claim 6, characterized in that the arbuscular mycorrhizal fungus is Glomus intraradices.

8. The process as claimed in claim 1, wherein an impact mill is used for comminuting the crude inoculum.

9. The process as claimed in claim 8, wherein the impact mill is a hammer mill or a bladed-disk mill or an air-jet mill.

10. The process as claimed in claim 8, wherein the operative grinding parameters at the mill are adjusted so that the ground product has an average particle size of 100-500 μm.

11. The process as claimed in claim 10, wherein the rotational speed of the mill is adjusted accordingly and its discharge screen is selected accordingly.

12. An arbuscular mycorrhizal fungal preparation, obtainable by the process as claimed in a claim 1.

13. A method of using the arbuscular mycorrhizal fungal preparation as claimed in claim 12 as complementation of fertilzation and crop protection in agriculture and silviculture.

14. The method as claimed in claim 13, characterized in that the arbuscular mycorrhizal fungal preparation is employed directly, either in solid form, or diluted with an inert substance or, together with formulation auxiliaries, in the for of a water-suspendable preparation.
